(19) United States
(12) Patent Application Publication
(10) Pub. No.: US 2019/0049458 A1
(43) Pub. Date: Feb. 14, 2019

(54) SYSTEMS, APPARATUSES, AND METHODS FOR ASSESSMENT OF LONG TERM STABILITY OF SAMPLES

(71) Applicant: UNCHAINED LABS, Pleasanton, CA (US)

(72) Inventors: Ernesto FREIRE, North Bethesda, MD (US); Richard BROWN, East Falmouth, MA (US)

(21) Appl. No.: 16/080,271

(22) PCT Filed: Feb. 28, 2017
(86) PCT No.: PCT/US17/19980

§ 371 Date: Aug. 27, 2018

Related U.S. Application Data
(60) Provisional application No. 62/301,085, filed on Feb. 29, 2016.

(51) Int. Cl. G01N 33/68; G01N 21/75; G01N 21/64

(52) U.S. Cl. CPC G01N 33/68; G01N 2021/755; G01N 21/6486; G01N 21/75

(57) ABSTRACT

A method includes receiving a sample. The method also includes applying a denaturing agent to a first portion of the sample, and detecting, in response to the application of the denaturing agent, a first measure from the first portion of the sample. The method also includes modifying the temperature of a second portion of the sample and detecting, in response to the modifying the temperature of the second portion of the sample, a second measure from the second portion of the sample. The method also includes computing thermodynamic information for the sample based on the indication of the first measure, and computing kinetic information for the sample based on the indication of the second measure. The method also includes computing, based on the thermodynamic information and the kinetic information, an indication of temporal stability of the protein component of the sample.

### CROSS-REFERENCE TO RELATED APPLICATIONS

This application claims priority to U.S. Provisional Application No. 62/301,085 filed Feb. 29, 2016, titled “PREDICTION OF LONG TERM STABILITY OF BIOLOGICS”, the entire disclosure of which is incorporated herein by reference.

### BACKGROUND

The formation of aggregates that originate from the presence of denatured protein is a significant problem in the formulation of biologics and detrimental to their long term stability. Quantitative predictive methods of long term stability are non-existent. At best, qualitative correlations that are not universally valid have been made between stability and physical parameters like denaturation temperature, Tm, or the onset of temperature induced aggregation, Tagg. Thus, there exists a need for methods and systems to quantitatively predict long term stability of protein compositions by integrating thermodynamic and kinetic information.

### SUMMARY

In some embodiments, a method includes receiving a sample, the sample including a protein component. The method also includes applying a denaturing agent to a first portion of the sample. The method also includes detecting, in response to the application of the denaturing agent, a first measure from the first portion of the sample. The first measure is indicative of a thermodynamic state of the first portion of the sample. The method also includes modifying the temperature of a second portion of the sample and detecting, in response to the modifying the temperature of the second portion of the sample, a second measure from the second portion of the sample. The second measure is indicative of a rate of denaturation of the protein in the second portion of the sample. The method also includes computing thermodynamic information for the sample based on the indication of the first measure, and computing kinetic information for the sample based on the indication of the second measure. The method also includes computing, based on the thermodynamic information and the kinetic information, an indication of temporal stability of the protein component of the sample.

In some embodiments, a method includes receiving a sample, the sample including a protein component. The method also includes applying a denaturing agent to a first portion of the sample. The method also includes detecting, in response to the application of the denaturing agent, a first measure from the first portion of the sample. The first measure is indicative of a thermodynamic state of the first portion of the sample. The method also includes modifying the temperature of a second portion of the sample and detecting, in response to the modifying the temperature of the second portion of the sample, a second measure from the second portion of the sample. The second measure is indicative of a rate of denaturation of the protein in the second portion of the sample. The method also includes computing thermodynamic information for the sample based on the indication of the first measure, and computing kinetic information for the sample based on the indication of the second measure. The method also includes computing, based on the thermodynamic information and the kinetic information, an indication of temporal stability of the protein component of the sample.

In some embodiments, a system includes a sample holder configured to receive a sample, the sample including a protein component. The system also includes a first apparatus configured to receive a first portion of the sample, the first apparatus configured to apply a denaturing agent to the first portion of the sample, and to detect, in response to the application of the denaturing agent, a first measure from the first portion of the sample. The first measure is indicative of a thermodynamic state of the first portion of the sample. The system also includes a second apparatus configured to receive a second portion of the sample, the second apparatus configured to modify the temperature of the second portion of the sample, and to detect, in response to the modifying the temperature of the second portion of the sample, a second measure from the second portion of the sample. The second measure is indicative of a rate of denaturation of the protein in the second portion of the sample.

The system also includes a processor communicably coupled to the first apparatus and the second apparatus, the processor configured to receive an indication of the first measure, and to compute thermodynamic information for the sample based on the indication of the first measure. The processor is also configured to receive an indication of the second measure, and to compute kinetic information for the sample based on the indication of the second measure. The processor is further configured to, based on the thermodynamic information and the kinetic information, compute an indication of temporal stability of the protein component of the sample.

### BRIEF DESCRIPTION OF THE DRAWINGS

The above and further features will be more clearly appreciated from the following detailed description when taken in conjunction with the accompanying drawings.

FIG. 1 is an illustration of a system for assessment of long term stability of biologics, according to embodiments.

FIG. 2 is a flow chart of a method for assessment of long term stability of biologics, according to embodiments.

FIG. 3 is a graph showing that isothermal chemical denaturation (ICD) allows experimental determination of the thermodynamic parameters that describe the native/denatured equilibrium of a protein.

FIG. 4 is a graph showing temperature denaturation measured by change in fluorescence emission. Analysis of the data provides the rate constant and its temperature dependence.

FIG. 5 includes graphs showing that an intrinsic rate constant can be determined directly by measuring denaturation as a function of time at different temperatures below the denaturation temperature (right panel). The rate will decrease at lower temperatures. The temperature dependence of the rate provides the necessary information to calculate the rate at lower temperatures (e.g., 25° C.).

FIG. 6 includes graphs showing that an Arrhenius plot of the rates measured at different temperatures provides the energy of activation (ΔH*) and its temperature dependence (ΔCp).

FIG. 7 is a graph showing that a plot of

$$\frac{1}{{F}_{M}^{n - 1}}$$

versus time should yield a straight line if the selected n value is the one that best accounts for the experimental data.

FIG. 8 is a graph showing the effect of ΔG on aggregation for the same kinetic rate at a constant protein concentration.

FIG. 9 is a graph showing the effect of kinetic rate on aggregation for the same ΔG at a constant protein concentration.

### DETAILED DESCRIPTION

As used herein “denaturation” is a process in which proteins lose the quaternary structure, tertiary structure and secondary structure which are present in their native state, by application of some external stress or reagent such as a strong acid or base, a concentrated inorganic salt, an organic solvent, radiation or heat. Commonly used chemical denaturants include urea and guanidine hydrochloride. Protein denaturation in living cells may result in disruption of cellular activity and possibly cell death. In vivo or in vitro, denatured proteins can exhibit a wide range of characteristics, from conformational change and loss of solubility to aggregation due to the exposure of hydrophobic groups. In this process, exposed hydrophobic portions of the unfolded protein may interact with the exposed hydrophobic patches of other unfolded proteins, spontaneously leading to protein aggregation.

As used herein, the singular forms “a,” “an” and “the” include plural referents unless the context clearly dictates otherwise.

Aspects disclosed herein are directed to systems, apparatuses, and methods that are useful to quantitatively predict long term stability (i.e., over time) of a solution by integrating thermodynamic information and kinetic information and, with respect to the appearance, growth, and accumulation of denatured state aggregates. For purposes of technical explanation, and without being limited by any particular theory, the rate of aggregation of a biologic molecule such as a protein is proportional to two different parameters: (1) the concentration of aggregating species and (2) the intrinsic rate at which they aggregate.

Denatured state aggregation can be described by the following set of relations:

$$\begin{matrix}N {⇌}^{K} D & ( 1 ) \\ nD \to {D}_{n} & ( 2 )\end{matrix}$$

A protein in solution in its native state N exists in equilibrium with its denatured state D. The denatured state has a tendency to self-associate and form aggregates. In Equation 2, n is the average stoichiometry of the aggregates. These aggregates are usually reversible in their early stages but eventually become irreversible. As aggregates start to accumulate, the equilibrium in Equation 1 is restored by the generation of additional denatured protein. Irreversible aggregates essentially act as a sink, eventually leading to the disappearance of the native state. In protein solutions (e.g., biologics and therapeutic solutions), well before the native state disappears (e.g., about 5% aggregation or less) the protein solution becomes useless due to safety and potency considerations. Other denatured aggregation models are also possible. Generally, all of the aggregation models depend on at least two parameters: the concentration of denatured protein and the intrinsic rate of aggregation. A simple n^{th} order rate equation can describe aggregate formation in most situations:

$$\frac{d [ M ]}{dt} = - {nk [ D ]}^{n} \qquad ( 3 )$$

In the Equation 3, [M] is the concentration of protein in monomeric form [M]=[N]+[D] and k is the rate constant for aggregate formation. The concentration of denatured protein can be written as [D]=F_{D}[M], and Equation 1 becomes:

$$\frac{d [ M ]}{dt} = - {{nkF}_{D}^{n} [ M ]}^{n} = - {{k}_{obs} [ M ]}^{n} \qquad ( 4 )$$

where k_{obs}=nkF_{D}ⁿ. The general solution to Equation 4 becomes:

$$\frac{1}{{[ M ]}^{n - 1}} - \frac{1}{{[ M ]}_{o}^{n - 1}} = ( n - 1 ) {k}_{obs} t \qquad ( 5 )$$

where [M]ₒ is the concentration of monomers at time zero. Equation 5 can be rearranged as:

$${F}_{M}^{n - 1} = \frac{1}{1 + {( n - 1 ) [ {M}_{o} ]}^{n - 1} {k}_{obs} t} \qquad ( 6 )$$

where

$${F}_{M} = \frac{[ M ]}{{[ M ]}_{o}} .$$

Equation 6 describes the disappearance of monomers with time. This relation depends on the concentration of monomers at time zero, which is essentially equal to the total protein concentration and the observed rate constant, k_{obs}. The protein concentration is known and k_{obs} depends on two quantities: the fraction of denatured protein among the monomeric protein, F_{D}, and the intrinsic rate constant k. In some embodiments, and as laid out in more detail herein, these two quantities, as well as n, can be determined by a combination of isothermal chemical denaturation (ICD) and temperature denaturation experiments.

Experimental Determination of F_{D}

Protein monomers include the native and denatured states, which exist in thermodynamic equilibrium [M]=[N]+[D]. Equation 1 corresponds to the equilibrium between native and denatured states observed at low temperatures (e.g., room temperature of 25° C., or less). Equation 1 is also referred to as the two-state native/denatured equilibrium characteristic of single domain proteins or cooperative domains in multi-domain proteins. The equilibrium constant, K, is determined by the magnitude of the Gibbs energy, ΔG:

$$K = {e}^{- \frac{Δ G}{RT}} \qquad ( 7 )$$

where T is the absolute temperature and R the gas constant. The fraction of denatured, F_{D}, protein (or denatured domain) is given by:

$${F}_{D} = \frac{K}{1 + K} = \frac{{e}^{- \frac{Δ G}{RT}}}{1 + {e}^{- \frac{Δ G}{RT}}} \qquad ( 8 )$$

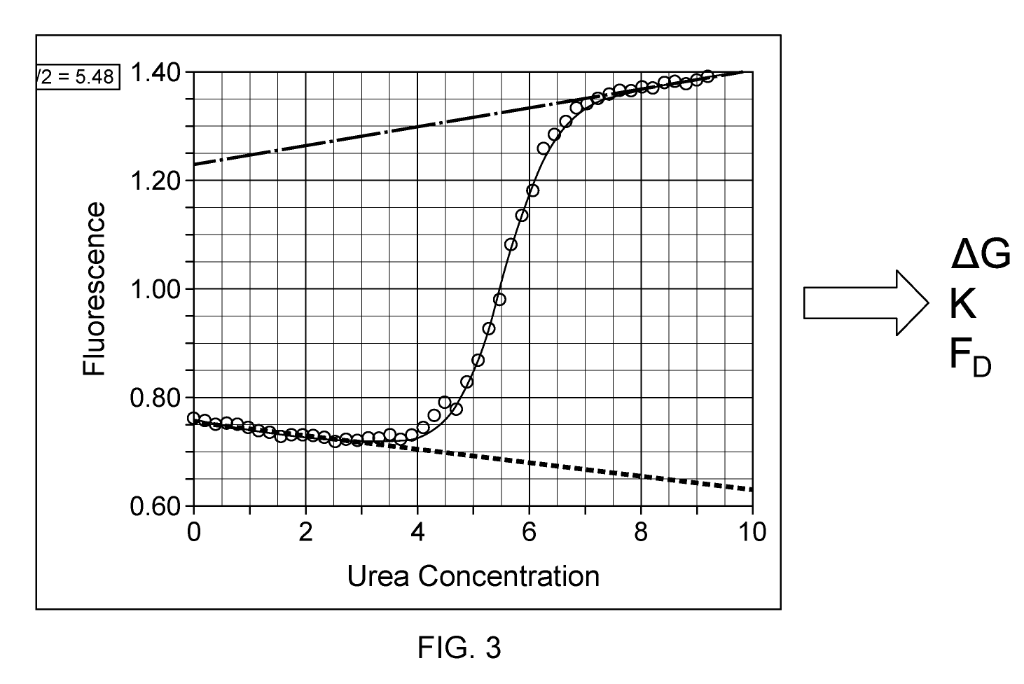

This is the quantity required to solve Equation 6. The fraction of native protein, F_{N}, is simply F_{N}=1−F_{D}. As best illustrated in FIG. 3, one way to determine K and ΔG is by isothermal chemical denaturation (ICD). As illustrated in FIG. 3, fluorescence data is collected at a series of urea concentrations for a sample, and the raw data is fit to a protein unfolding transition state model (not shown). ΔG can be determined by nonlinear least-squares fitting of the data to the model. K can then be determined using Equation (7). Typically, ΔG increases with the stability of a sample including protein.

Experimental Determination of the Rate Constant

The intrinsic rate constant, k, can be measured in at least two different forms. The first form is from the shape of the temperature denaturation profile of the protein. The temperature denaturation profile can be measured by different techniques, including but not limited to, differential scanning fluorescence (DSF) and differential scanning calorimetry (DSC). Since temperature denaturation is usually coupled to aggregation that can be measured by, for example, dynamic light scattering, the following equations can also be applied to temperature aggregation data. The irreversible temperature denaturation of proteins is kinetically controlled and that analysis of the shape of the transition profile provides the rate constant and the temperature dependence of the rate in terms of the activation enthalpy, entropy, and heat capacity (ΔH*, ΔS*, and ΔCₚ*).

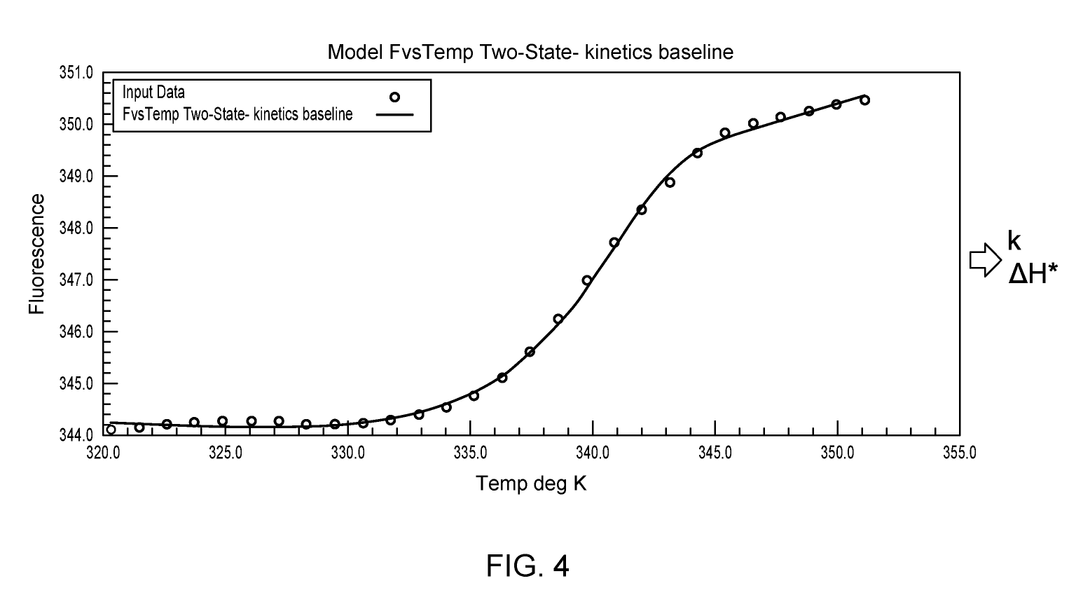

The temperature denaturation curve (FIG. 4) can be quantitatively accounted for by the following equation for F_{D}:

$${F}_{D} = 1 - exp ( - exp ( - \frac{Δ {G}^{*}}{RT} ) ) \qquad ( 9 )$$

where ΔG* is the activation free energy (ΔG*=ΔH*−T ΔS*). The temperature dependence of ΔH* and ΔS* are defined in terms of ΔCp*: ΔH*=ΔH*(T_{R})+ΔCp*(T−T_{R}) and ΔS*=ΔS*(T_{R})+ΔCp*ln(T/T_{R}), where T_{R} is the reference temperature. The intrinsic rate constant, k, is:

$$k = exp ( - \frac{Δ {G}^{*}}{RT} ) \qquad ( 10 )$$

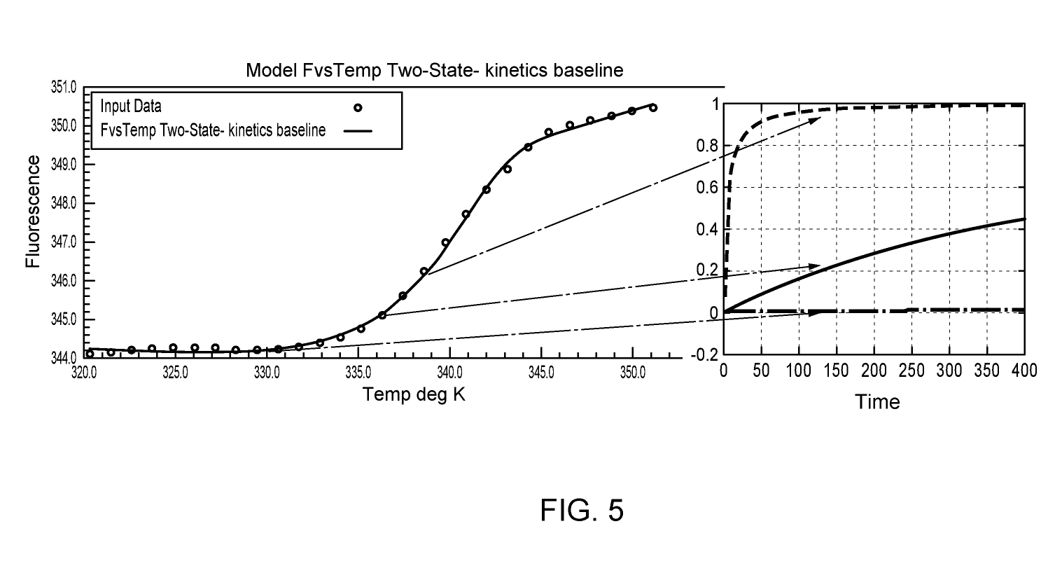

A second example method to determine the intrinsic rate constant is by measuring protein denaturation as a function of time at constant temperatures below the denaturation transition. See, FIG. 5.

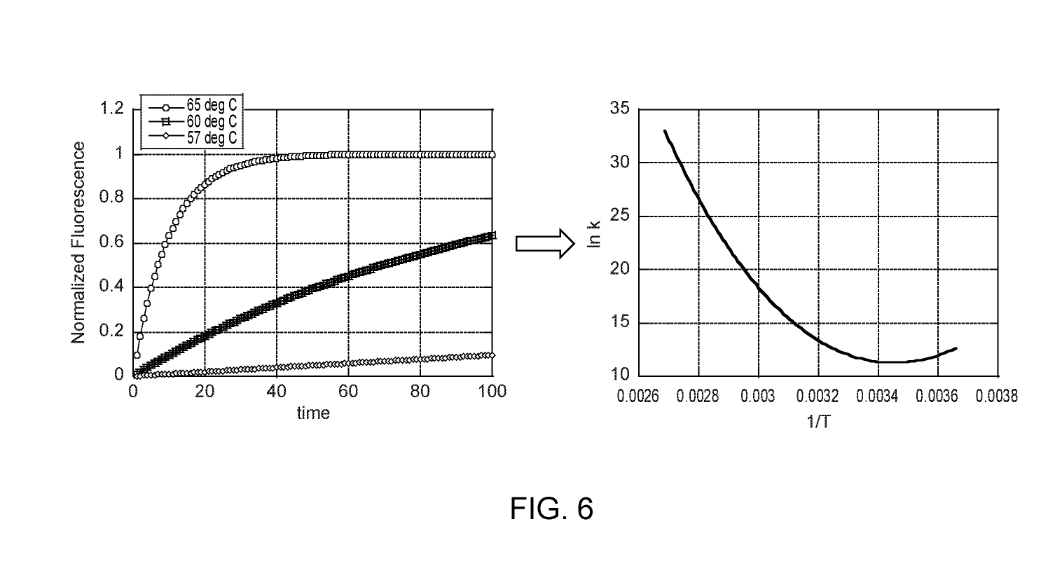

In the right panel of FIG. 5, the normalized expected change in fluorescence at three different temperatures is shown. As seen in the figure, at temperatures close to the transition midpoint, the change is very fast with the rate of change becomes progressively slower at lower temperatures. At room temperature (i.e., 25° C.) the change may take weeks to months. A quick assessment of the expected rate at room temperature or below room temperature requires extrapolation of the rates obtained at higher temperatures, at which denaturation is faster and can be accurately measured. This can be done by performing an Arrhenius analysis of the data, as shown in FIG. 6.

FIG. 6 shows that an Arrhenius analysis of the rates obtained at different temperatures yields the activation energy, entropy and heat capacity:

$$lnk = - \frac{Δ {G}^{*}}{RT} = - \frac{Δ {H}^{*}}{RT} + \frac{Δ {S}^{*}}{R} = - \frac{Δ {H}^{*} ( {T}_{R} ) + Δ {C}_{p}^{*} ( T - {T}_{R} )}{RT} + \frac{Δ {S}^{*} ( {T}_{R} ) + Δ {C}_{p}^{*} ln ( T \text{/} {T}_{R} )}{R} \qquad ( 11 )$$

Equation 11 allows calculation of the intrinsic rate at any desired temperature.

Determination of the Average Stoichiometry of Aggregates

Equation 5 is the standard integrated form of an n^{th} order rate equation. A plot of

$$\frac{1}{{[ M ]}^{n - 1}} or \frac{1}{{F}_{M}^{n - 1}}$$

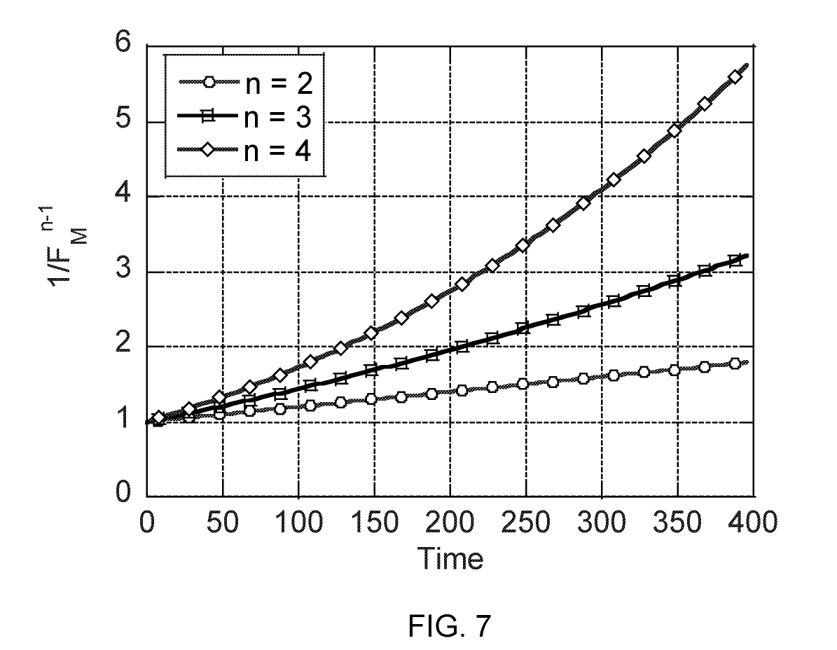

versus time should result in a straight line. FIG. 7 illustrates the situation for the case in which dimers define the average stoichiometry of the aggregates. A plot with n=2 yields a straight line. Non-linear least squares of the experimental data is used to simultaneously determine the rate constant, k, and the stoichiometry value, n.

Accordingly, in some embodiments, a combination of isothermal chemical denaturation and temperature denaturation experiments can provide parameters to calculate the fraction of protein in monomeric form, F_{M}, or the fraction of aggregated protein as a function of time as defined by Equation 6. While Equation 3 corresponds to a specific, example model, other aggregation models can also be considered as the analysis presented here provides two parameters for any model: (1) The concentration of denatured aggregating species and (2) the intrinsic rate of denaturation/aggregation as a function of temperature.

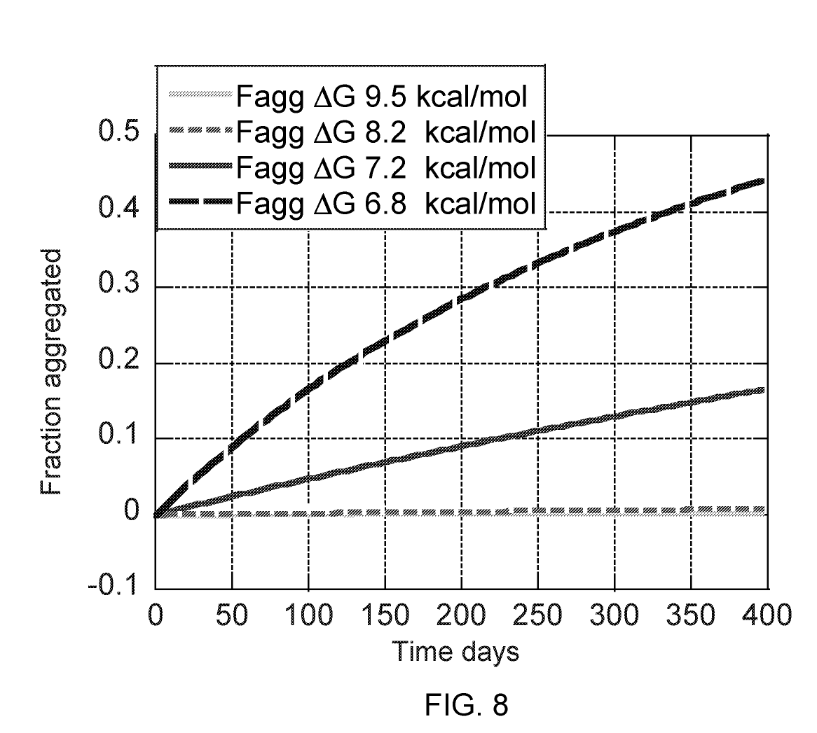

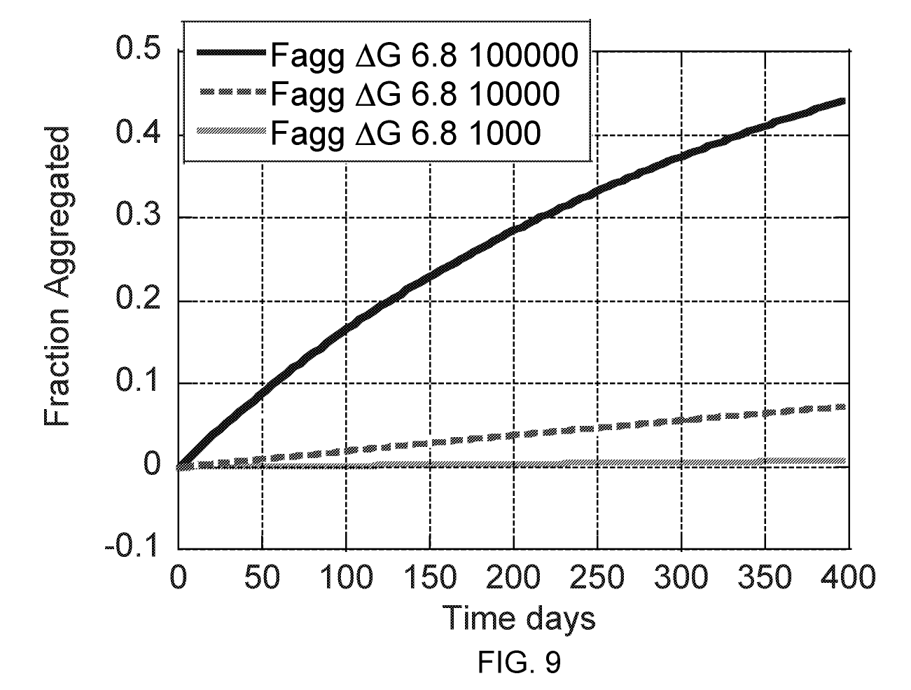

FIG. 8 illustrates the effects of ΔG for the native/denatured equilibrium, which determines the concentration of denatured aggregating species, for a constant intrinsic rate. FIG. 9 illustrates the effects of the intrinsic rate for a constant ΔG value. Independent of a specific model, maximal long term stability will be achieved when the concentration of aggregating species is minimal (maximal ΔG) and the intrinsic rate of aggregation (k) is minimal. The combination of isothermal chemical denaturation and temperature denaturation experiments provide both parameters. FIGS. 8, 9 indicate that maximizing ΔG and minimizing k yield the longest long term stability. In FIGS. 8, 9, the protein concentration was about 100 mg/ml.

Aspects disclosed herein illustrate that, at any given temperature, the time course of protein aggregation is a function of thermodynamic and kinetic parameters: the concentration of aggregating species at time zero (which depends on protein concentration and ΔG) and the rate of protein aggregation (k).

Systems and Apparatuses

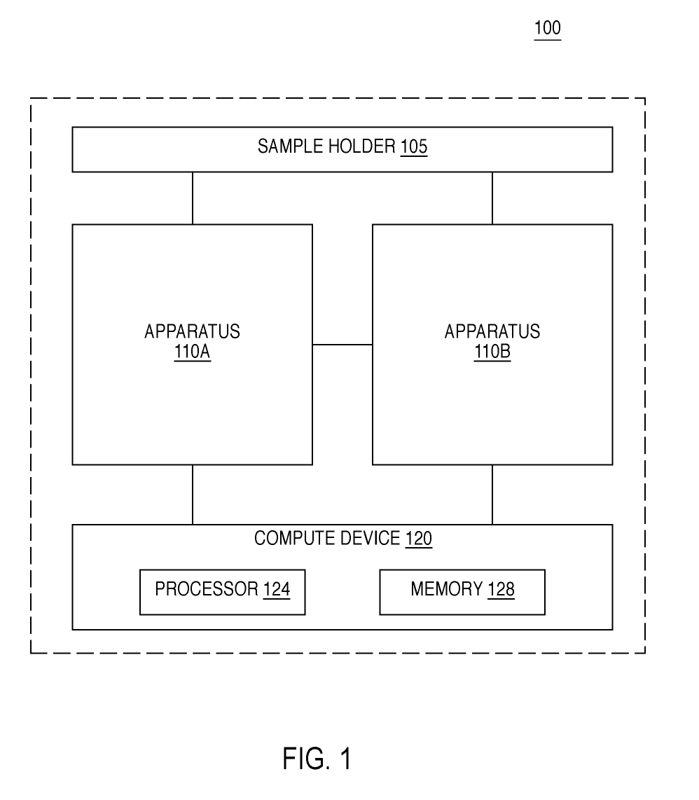

FIG. 1 is a schematic diagram that illustrates a system 100 configured for assessment of long term stability (also sometimes referred to as “temporal stability”) of a sample, such as, for example, a sample containing biologics such as protein. The system 100 can include a sample holder 105, an apparatus 110A (also sometimes referred to as a “first apparatus”), an apparatus 110B (also sometimes referred to as a “second apparatus”), and a compute device 120. In some embodiments, one or more of the components of the system 100 can be within the same housing. In some embodiments, all the components of the system 100 can be within the same housing.

The compute device 120 can be, for example, a server, a compute device, a router, a data storage device, a tablet, a mobile device, and/or the like. The compute device 190 can include, for example, computer software (stored in and/or executed at hardware) such as a web application, a database application, a cache server application, a queue server application, an operating system, a file system, and/or the like; computer hardware such as a network appliance, a storage device (e.g., disk drive, memory module), a processing device (e.g., computer central processing unit (CPU)), a computer graphic processing unit (GPU), a networking device (e.g., network interface card), and/or the like; and/or combinations of computer software and hardware. In some instances, although not shown in FIG. 1, the compute device 105 can be operatively coupled to more other apparatuses and/or devices.

As shown in FIG. 1, the compute device 120 includes a memory 128 and a processor 124, and can include other component(s) (not shown in FIG. 1), such as, for example, an interface to permit a user/operator to interact with the compute device 120 and/or the system 100. The memory 128 can be, for example, a Random-Access Memory (RAM) (e.g., a dynamic RAM, a static RAM), a flash memory, a removable memory, and/or so forth. In some instances, instructions associated with performing the operations described herein (e.g., computing temporal stability) can be stored within the memory 128 and executed at the processor 124.

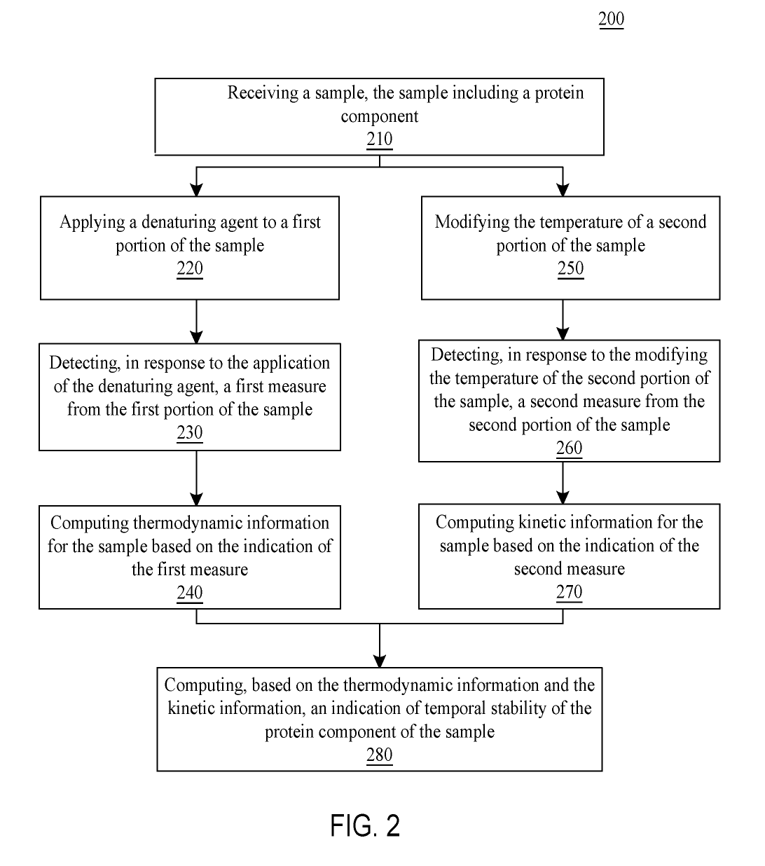

In some embodiments, each module/component in the processor 124 can be any combination of a hardware-based module/component (e.g., a field-programmable gate array (FPGA), an application specific integrated circuit (ASIC), a digital signal processor (DSP)), a software-based module/component (e.g., a module of computer code stored in the memory 128 and/or executed at the processor 124), and/or a combination of hardware- and software-based modules/components. Each module/component in the processor 124 is capable of performing one or more functions/operations such as those described in further detail with respect to FIGS. 2-9. In some instances, the modules/components included and executed in the processor 124 can be, for example, a process, an application, a virtual machine, and/or some other hardware or software module/component (stored in memory and/or executing in hardware). The processor 124 can be any suitable processor configured to run and/or execute such modules/components.

In other instances, the processor 124 can be configured for performing operations other than those described with respect to FIGS. 1-2. For example, the processor 124 can be configured for simultaneously performing multiple computing tasks for multiple systems and/or processes. In some instances, the compute device 120 can include more components than those shown in FIG. 1. For example, the compute device 120 can include a communication interface (e.g., a data port, a wireless transceiver and an antenna) to enable data transmission between the compute device 120 and other devices and/or the apparatuses 110A, 110B. In some instances, the compute device 120 can include or be coupled to a display device (e.g., a printer, a monitor, a speaker, etc.), such that an output of the compute device can be presented to a user 170 via the display device.

In some embodiments, the compute device 120 and/or the system 100 can be operatively coupled to other devices via, for example, a network. The network can be any type of network that can operatively connect and enable electronic transmission therebetween. The network can be, for example, a wired network (an Ethernet, local area network (LAN), etc.), a wireless network (e.g., a wireless local area network (WLAN), a Wi-Fi network, etc.), or a combination of wired and wireless networks (e.g., the Internet, etc.).

In some embodiments, the sample holder 105 is configured to receive a sample. The sample holder 105 can include any suitable receiving interface for receiving the sample and/or a container including the sample, such as, but not limited to, a cartridge holder, a well plate such as a microtiter plate, a test tube holder, and/or the like. In some embodiments, the sample holder 105, or a portion thereof, may be integrally or removably formed with the first apparatus 110A and/or the second apparatus 110B. For example, the sample holder 105 can include a microtiter plate that can be snapped into place in an appropriate receptacle in the apparatus 110A. In some embodiments, the sample includes a protein component, such as any suitable amino acid sequence.

In some embodiments, the first apparatus 110A is configured to receive a first portion of the sample from the sample holder 105 via any suitable means. For example, in some embodiments, the first portion of the sample is transferred to the first device via passive means (e.g., gravity-driven flow), via active means (e.g., using a pump), or both. In some embodiments, a metering pump can be employed to transfer a precise amount of sample as the first portion from the sample holder 105 to the first apparatus 110A.

In some embodiments, the first apparatus 110A is further configured to apply a denaturing agent to the first portion of the sample. For example, in some embodiments, the first apparatus 110A includes or is fluidly coupled to a source of denaturing agent, such as urea. In some embodiments, the denaturing agent can be one or more of suitable acids such as acetic acid, bases such as sodium bicarbonate, solvents such as ethanol, cross-linking agents such as formaldehyde, chaotropic agents such as urea, disulfide bond reducers such as 2-mercaptoethanol, and/or the like. In some embodiments, the first device includes fluid handling means, such as a pump and tubing, to deliver the denaturing agent to the first portion of the sample. In some embodiments, the first device includes fluid metering means, such as a metering pump, to delivery a predetermined quantity of the denaturing agent to the first portion of the sample.

In some embodiments, the first apparatus 110A is further configured to detect, in response to the application of the denaturing agent, a first measure from the first portion of the sample. In some embodiments, the first apparatus 110A is further configured to excite the first portion of the sample with an excitation light (e.g., from a suitable light source such as LEDs, a laser, and/or the like), and detect, in response to the application of the denaturing agent and in response to the excitation, the first measure from the first portion of the sample. For example, in some embodiments, the first apparatus 110A can be configured for excitation and fluorescence detection at wavelengths corresponding to intrinsically fluorescent amino acids such as tryptophan, tyrosine, and phenylalanine. In some embodiments, the first measure is indicative of a thermodynamic state of the first portion of the sample. In some embodiments, the first measure includes fluorescence intensity, and/or a derivative thereof (e.g., scaled fluoresence intensity, fluorescence lifetime, and/or the like).

In some embodiments, the first apparatus 110A includes a set of compartments (e.g., a well plate) configured to hold the first portion of the sample. In some embodiments, the first apparatus 110A is configured to aliquot the first portion of the sample into the set of compartments. In some embodiments, the sample holder 105 is configured to aliquot the first portion of the sample into the set of compartments. In some embodiments, the sample holder 105 includes the set of compartments. In such embodiments, the first apparatus 110A is further configured to apply the denaturing agent to the first portion of the sample by applying a different quantity of the denaturing agent to the sample held in each compartment of the set of compartments. Said another way, the first apparatus 110A is configured to treat the sample in each compartment to a different level of the denaturing agent. In such embodiments, the first measure is one of a set of first measures, and the first apparatus 110A is further configured to detect the first measure by detecting the set of first measures. Each measure of the set of first measures corresponds to a different compartment of the set of compartments. In this manner, the first apparatus 110A can be configured to collect fluorescence data from each compartment to generate the plot illustrated in FIG. 3.

In some embodiments, the first apparatus 110A is configured to maintain the first portion of the sample at a substantially constant temperature. For example, in some embodiments, the first apparatus 110A includes a heating element (e.g., heat block, a heat plate, a heating coil, and/or the like), in contact with, or in the proximity of, the first portion of the sample. In some embodiments, the processor 124 of the compute device 120 is configured to control the current delivered to the heating element (e.g., via a drive circuit) to establish and maintain the temperature of the heating element and the first portion of the sample.

In some embodiments, the first apparatus 110A, and its various components described herein, are collectively configured to detect the first measure by isothermal chemical denaturation (ICD), i.e., any suitable chemical denaturation approach carried out while maintaining the sample at a substantially constant temperature.

Still referring to FIG. 1, in some embodiments, the second apparatus 110B is configured to receive a second portion of the sample from the sample holder 105. In some embodiments, the second apparatus 110B is further configured to modify the temperature of the second portion of the sample. For example, in some embodiments, the second apparatus 110B includes a heating element (e.g., a heat block, a heat plate, a heating coil, and/or the like), in contact with, or in the proximity of, the second portion of the sample. In some embodiments, the processor 124 of the compute device 120 is configured to control the current delivered to the heating element (e.g., via a drive circuit) to vary the temperature of the heating element (e.g., in a continuous or stepwise manner) and the second portion of the sample.

In some embodiments, the second apparatus 110B is further configured to detect, in response to the modifying the temperature of the second portion of the sample, a second measure from the second portion of the sample.

In some embodiments, the second apparatus 110B, and its various components described herein, are collectively configured to detect the second measure by temperature denaturation scanning.

In some embodiments, the second apparatus 110B, and its various components described herein, are collectively configured to detect the second measure by single temperature denaturation kinetics.

In some embodiments, the second measure indicative of a rate of denaturation of the protein in the second portion of the sample. In some embodiments, the second measure includes fluorescence intensity and/or a derivative thereof.

Still referring to FIG. 1, in some embodiments, the processor 124 of the compute device 120 is configured to receive an indication of the first measure from the apparatus 110A, and is further configured to receive an indication of the second measure from the apparatus 110B. In some embodiments, the processor 124 is further configured to compute thermodynamic information for the sample based on the indication of the first measure. For example, as discussed herein with respect to equations 6-8, the processor 124 can be configured to compute one or more of F_{D}, F_{N}, K and/or ΔG for the sample based on the first measure. Generally, in some embodiments, the processor 124 is configured to compute the thermodynamic information by estimating an equilibrium constant associated with the denaturing of the protein in the first portion of the sample, and/or a free enthalpy associated with the denaturing of the protein in the first portion of the sample.

In embodiments where the first apparatus 110A includes a set of compartments as described herein, the processor 124 can be configured to receiving an indication of the set of first measures, and to compute the thermodynamic information by computing thermodynamic information based on the indication of the set of first measures.

In some embodiments, the processor 124 is further configured to compute kinetic information for the sample based on the indication of the second measure. For example, as discussed herein with respect to equations 9-10, the processor 124 can be configured to compute one or more of ΔG* and k for the sample based on the second measure. Generally, in some embodiments, the processor 124 is configured to compute the kinetic information by estimating a standard enthalpy of activation associated with denaturing induced by the modifying the temperature of the second portion of the sample.

In some embodiments, the processor 124 is further configured to compute, based on the thermodynamic information and the kinetic information, an indication of temporal stability (i.e., stability over time) of the protein component of the sample. For example, as discussed herein with respect to equation 4, in some embodiments, the processor 124 can be configured to compute k_{obs} for the sample. In some embodiments, the processor 124 is configured to compute the indication of temporal stability by computing a product of the thermodynamic information and the kinetic information. In some embodiments, the indication of temporal stability is a denaturation rate associated with the protein in the sample.

In some embodiments, the processor 124 is further configured to deem the sample to be a pharmaceutically acceptable protein composition if the indication of temporal stability meets a predetermined criterion. In some embodiments, the sample meets the predetermined criterion when the indication of temporal stability is lower than a predetermined threshold, e.g., is lower than a predetermined value of denaturation rate. In some embodiments, the sample meets the predetermined criterion when the indication of temporal stability is within a predetermined range. In some embodiments, the sample meets the predetermined criterion when the indication of temporal stability is greater than or equal to a predetermined threshold.

FIG. 2 illustrates a method 200 according to some embodiments. The method 200 can be executed at least in part by the system 100, or a system that is structurally and/or functionally similar thereto. Described with respect to the system 100 for simplicity, in some embodiments, the method 200 includes, at step 210, receiving a sample (e.g., at the sample holder 105), the sample including a protein component. The method 200 also includes, at 220, applying a denaturing agent to a first portion of the sample (e.g., at the first apparatus 110A). In some embodiments, the denaturing agent includes urea. In some embodiments, the method 200 further includes maintaining the first portion of the sample at a substantially constant temperature.

The method 200 also includes, at 230, detecting, in response to the application of the denaturing agent, a first measure from the first portion of the sample. In some embodiments, the first measure is indicative of a thermodynamic state of the first portion of the sample. In some embodiments, the first measure includes fluorescence. In some embodiments, the detecting at step 230 includes detecting the first measure via isothermal chemical denaturation (ICD).

The method 200 also includes, at 240, computing thermodynamic information for the sample based on the indication of the first measure (e.g., via the processor 124). In some embodiments, the computing at step 240 further includes estimating one or more of: an equilibrium constant associated with the denaturing of the protein in the first portion of the sample; and a free enthalpy associated with the denaturing of the protein in the first portion of the sample.

In some embodiments, the method 200 also includes aliquoting the first portion of the sample into a set of compartments of a first device. In such embodiments, the applying at step 220 can further include applying a different quantity of the denaturing agent to each compartment of the set of compartments. In such embodiments, the detecting at step 230 can further include detecting a set of first measures, each measure of the set of first measure corresponding to a different compartment of the set of compartments. In such embodiments, the computing at step 240 can further include computing thermodynamic information based on the indication of the set of first measures.

The method 200 also includes, at 250, modifying the temperature of a second portion of the sample (e.g., via the second apparatus 110B).

The method 200 also includes, at 260, detecting, in response to the modifying the temperature of the second portion of the sample, a second measure from the second portion of the sample. In some embodiments, the second measure includes fluorescence. In some embodiments, the detecting at step 260 further includes detecting the second measure via temperature denaturation scan or by single temperature denaturation kinetics.

The method 200 also includes, at 270, computing kinetic information for the sample based on the indication of the second measure. In some embodiments, the computing at step 270 includes estimating a standard enthalpy of activation associated with denaturing induced by the modifying the temperature of the second portion of the sample.

The method 200 also includes, at 280, computing, based on the thermodynamic information and the kinetic information, an indication of temporal stability of the protein component of the sample. In some embodiments, the computing at step 280 further includes computing a product of the thermodynamic information and the kinetic information. In some embodiments, the indication of temporal stability is a denaturation rate associated with the protein in the sample.

In some embodiments, the method 200 further includes deeming the sample to be a pharmaceutically acceptable protein composition if the indication of temporal stability meets a predetermined criterion (e.g., is above/below a predetermined threshold, or is within a predetermined range).

In some embodiments, aspects disclosed herein are directed to a pharmaceutically acceptable protein composition as generated by the method 200 and/or the system 100.

In some embodiments, the systems, apparatus(es), and methods disclosed herein are useful for measuring dissociation constants as disclosed in U.S. Pat. No. 8,859,295 filed Aug. 22, 2011, titled “SYSTEM AND METHOD TO MEASURE DISSOCIATION CONSTANTS”, the entire disclosure of which is incorporated herein by reference in its entirety.

In some embodiments, the systems, apparatus(es), and methods disclosed herein are useful for creation of formulations and generation of denaturation graphs as disclosed in U.S. Pat. No. 8,609,040 filed Aug. 22, 2011, titled “SYSTEM FOR CREATION OF FORMULATIONS AND GENERATION OF DENATURATION GRAPHS”, the entire disclosure of which is incorporated herein by reference in its entirety.

In some embodiments, the systems, apparatus(es), and methods disclosed herein are useful for generating automated denaturation graphs as disclosed in U.S. Pat. No. 9,029,163 filed Aug. 22, 2011, titled “METHOD FOR GENERATION OF AUTOMATED DENATURATION GRAPHS”, the entire disclosure of which is incorporated herein by reference in its entirety.

In some embodiments, the systems, apparatus(es), and methods disclosed herein are useful for creating buffer solutions having a desired pH value as disclosed in U.S. Patent Application Publication No. 2012/0045846 filed Aug. 22, 2011, titled “SYSTEM AND METHOD FOR pH FORMULATIONS”, the entire disclosure of which is incorporated herein by reference in its entirety.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Examples of computer code include, but are not limited to, micro-code or micro-instructions, machine instructions, such as produced by a compiler, code used to produce a web service, and files containing higher-level instructions that are executed by a computer using an interpreter. For example, embodiments may be implemented using Java, C++, .NET, or other programming languages (e.g., object-oriented programming languages) and development tools. Additional examples of computer code include, but are not limited to, control signals, encrypted code, and compressed code.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

References, the entire disclosures of which are incorporated herein.
Arosio, P. et al. (2011) Aggregation Stability of a Monoclonal Antibody During Downstream Processing. Pharmaceutical Research 28, 1884-1894
Li, Y. and Roberts., C. J. (2010) Protein Aggregation Pathways, Kinetics, and Thermodynamics. In Aggregation of Therapeutic Proteins (Yang, W. and Roberts, C. J., eds.), pp. 63-102, Wiley
Greene, R. F., Jr. and Pace, C. N. (1974) Urea and guanidine hydrochloride denaturation of ribonuclease, lysozyme, alpha-chymotrypsin, and beta-lactoglobulin. J. Biol. Chem. 249, 5388-5393
Bolen, D. W. and Santoro, M. M. (1988) Unfolding free energy changes determined by the linear extrapolation method. 2. Incorporation of dGn-u values in a thermodynamic cycle. Biochemistry 27, 8069-8074
Santoro, M. M. and Bolen, D. W. (1988) Unfolding free energy changes determined by the linear extrapolation method. 1. Unfolding of phenylmethanesulfonyl.alpha.chymotrypsin using different denaturants. Biochemistry 27, 8063-8068
Freire, E. et al. (2013) Chemical denaturation as a tool in the formulation optimization of biologics. Drug Discovery Today 18, 1007-1013
Schon, A. et al. (2015) Denatured State Aggregation Parameters Derived from Concentration Dependence of Protein Stability. Analytical Biochemistry 488, 45-50
Sanchez-Ruiz, J. M. et al. (1988) Differential Scanning calorimetry of the Irreversible Thermal Denaturation of Thermolysin. Biochemistry 27, 1648-1652
Freire, E. et al. (1990) calorimetrically Determined Dynamics of Complex Unfolding Transitions in Proteins. Annu. Rev. Biophys. Biophys. Chem. 19, 159-188
Sanchez-Ruiz, J. M. (1992) Theoretical analysis of Lumry-Eyring models min differential scanning calorimetry. Biophys J 61, 921-935

What is claimed is:

1. A method, comprising:
receiving a sample, the sample including a protein component;
applying a denaturing agent to a first portion of the sample;
detecting, in response to the application of the denaturing agent, a first measure from the first portion of the sample, the first measure indicative of a thermodynamic state of the first portion of the sample;
modifying the temperature of a second portion of the sample;
detecting, in response to the modifying the temperature of the second portion of the sample, a second measure from the second portion of the sample, the second measure indicative of a rate of denaturation of the protein in the second portion of the sample;
computing thermodynamic information for the sample based on the indication of the first measure;
computing kinetic information for the sample based on the indication of the second measure; and
computing, based on the thermodynamic information and the kinetic information, an indication of temporal stability of the protein component of the sample.

2. The method of claim 1, further comprising aliquoting the first portion of the sample into a set of compartments of a first apparatus,
the applying the denaturing agent including applying a different quantity of the denaturing agent to each compartment of the set of compartments,
the detecting the first measure including detecting a set of first measures, each measure of the set of first measure corresponding to a different compartment of the set of compartments,
the computing the thermodynamic information by computing thermodynamic information based on the indication of the set of first measures.

3. The method of claim 1, wherein the denaturing agent includes urea.

4. The method of claim 1, wherein the first measure includes fluorescence.

5. The method of claim 1, further comprising maintaining the first portion of the sample at a substantially constant temperature.

6. The method of claim 1, the computing the thermodynamic information including estimating one or more of: an equilibrium constant associated with the denaturing of the protein in the first portion of the sample; and a free enthalpy associated with the denaturing of the protein in the first portion of the sample.

7. The method of claim 1, wherein the second measure includes fluorescence.

8. The method of claim 1, the computing the kinetic information including estimating a standard enthalpy of activation associated with denaturing induced by the modifying the temperature of the second portion of the sample.

9. The method of claim 1, the computing the thermodynamic information including estimating one or more of:
an equilibrium constant associated with the denaturing of the protein in the first portion of the sample; and
a free enthalpy associated with the denaturing of the protein in the first portion of the sample,
the computing the kinetic information including estimating a standard enthalpy of activation associated with denaturing induced by the modifying the temperature of the second portion of the sample, and
the computing the indication of temporal stability including computing a product of the thermodynamic information and the kinetic information.

10. The method of claim 1, wherein the indication of temporal stability is a denaturation rate associated with the protein in the sample.

11. The method of claim 1, the detecting the first measure including detecting the first measure via isothermal chemical denaturation (ICD).

12. The method of claim 1, the detecting the second measure including detecting the second measure via temperature denaturation scan or by single temperature denaturation kinetics.

13. The method of claim 1, further comprising deeming the sample to be a pharmaceutically acceptable protein composition if the indication of temporal stability meets a predetermined criterion.

14. The pharmaceutically acceptable protein composition of claim 13.

15. A system, comprising:
a sample holder configured to receive a sample, the sample including a protein component;
a first apparatus configured to receive a first portion of the sample, the first apparatus configured to:
apply a denaturing agent to the first portion of the sample; and
detect, in response to the application of the denaturing agent, a first measure from the first portion of the sample, the first measure indicative of a thermodynamic state of the first portion of the sample;
a second apparatus configured to receive a second portion of the sample, the second apparatus configured to:
modify the temperature of the second portion of the sample; and
detect, in response to the modifying the temperature of the second portion of the sample, a second measure from the second portion of the sample, the second measure indicative of a rate of denaturation of the protein in the second portion of the sample;
a processor communicably coupled to the first apparatus and the second apparatus, the processor configured to:
receive an indication of the first measure;
compute thermodynamic information for the sample based on the indication of the first measure;
receive an indication of the second measure;
compute kinetic information for the sample based on the indication of the second measure; and
based on the thermodynamic information and the kinetic information, compute an indication of temporal stability of the protein component of the sample.

16. The system of claim 15, the first apparatus including a set of compartments configured to hold the first portion of the sample, the first apparatus configured to aliquot the first portion of the sample into the set of compartments,
the first apparatus further configured to apply the denaturing agent to the first portion of the sample by applying a different quantity of the denaturing agent to each compartment of the set of compartments,
the first measure being one of a set of first measures, the first apparatus further configured to detect the first measure by detecting the set of first measures, each measure of the set of first measures corresponding to a different compartment of the set of compartments,
the processor further configured to receive an indication of the first measure by receiving an indication of the set of first measures, and to compute the thermodynamic information by computing thermodynamic information based on the indication of the set of first measures.

17. The system of claim 15, wherein the denaturing agent includes urea.

18. The system of claim 15, wherein the first measure includes fluorescence.

19. The system of claim 15, wherein the first apparatus is configured to maintain the first portion of the sample at a substantially constant temperature.

20. The system of claim 15, the processor configured to compute the thermodynamic information by estimating one or more of: an equilibrium constant associated with the denaturing of the protein in the first portion of the sample; and a free enthalpy associated with the denaturing of the protein in the first portion of the sample.

21. The system of claim 15, wherein the second apparatus includes one or more heat blocks configured to modify the temperature of the second portion of the sample.

22. The system of claim 15, wherein the second measure includes fluorescence.

23. The system of claim 15, the processor configured to compute the kinetic information by estimating a standard enthalpy of activation associated with denaturing induced by the modifying the temperature of the second portion of the sample.

24. The system of claim 15, the processor further configured to:
compute the thermodynamic information by estimating one or more of:
an equilibrium constant associated with the denaturing of the protein in the first portion of the sample; and
a free enthalpy associated with the denaturing of the protein in the first portion of the sample;
compute the kinetic information by estimating a standard enthalpy of activation associated with denaturing induced by the modifying the temperature of the second portion of the sample; and
compute the indication of temporal stability by computing a product of the thermodynamic information and the kinetic information.

25. The system of claim 15, wherein the indication of temporal stability is a denaturation rate associated with the protein in the sample.

26. The system of claim 15, the first apparatus further configured to detect the first measure by isothermal chemical denaturation (ICD).

27. The system of claim 15, the second apparatus further configured to detect the second measure by temperature denaturation scan or by single temperature denaturation kinetics.
